(19) United States
(12) Patent Application Publication
(10) Pub. No.: US 2025/0027927 A1
(43) Pub. Date: Jan. 23, 2025

(54) BIOSENSOR AND METHODS OF IN-SITU MEASURING OF NUTRIENT CONTENT OF A CROP CULTIVATION SUBSTRATE

(71) Applicant: North Carolina State University, Raleigh, NC (US)

(72) Inventors: Chadi SAYDE, Raleigh, NC (US); Riley LAWSON, Raleigh, NC (US); Amy Michele GRUNDEN, Raleigh, NC (US)

(21) Appl. No.: 18/775,588

(22) Filed: Jul. 17, 2024

Related U.S. Application Data
(60) Provisional application No. 63/514,223, filed on Jul. 18, 2023.

(51) Int. Cl. G01N 33/24; C12N 11/14; C12Q 1/00; C12Q 1/26; C12Q 1/58; G01K 7/02; G01K 7/16

(52) U.S. Cl. CPC G01N 33/245; C12N 11/14; C12Q 1/005; C12Q 1/26; C12Q 1/58; G01K 7/02; G01K 7/16

(57) ABSTRACT

The subject matter described herein is directed to systems, detection devices and enzyme sensors for real-time determination of the concentration levels of nutrients in a crop cultivation substrate using thermal signals and change in enthalpy per unit of enzyme. Methods of using the systems, detection devices and enzyme sensors for agricultural applications are described.

### CROSS-REFERENCE TO RELATED APPLICATIONS

This application claims the benefit of priority to U.S. Provisional Application No. 63/514,223, filed on Jul. 18, 2023, which is herein incorporated by reference in its entirety.

### FIELD

The presently disclosed subject matter relates generally to biosensors and methods for the in situ detection of nutrients in crop cultivation substrates.

### BACKGROUND

Nutrient detection in the substrates where crops are produced is a challenge for the development of a more effective food supply cycle. However, most soil nutrient testing is currently conducted by post-extraction laboratory testing, which does not provide real-time feedback and is not suited for routine analysis. That is, when the nutrient concentration in the crop cultivation substrate is detected, the detection work is completed in the laboratory after the crop cultivation substrate is collected and transported to the laboratory. The detection can be complicated and the whole process is cumbersome, inconvenient and labor intensive. The results of any analysis of this process cannot be quickly obtained which does not provide real-time feedback. Also, the complexity of this type of process limits its use and is not suited for real-time or even frequent analysis.

When nitrogen-based fertilizer application rates are greater than consumption rates, excess fertilizer is often washed away from the soil and is a main source of groundwater pollution.

Accumulation of excess fertilizer nutrients in the groundwater of irrigated areas with heavy agricultural use, such as nitrates, contribute to adverse environmental impacts including eutrophication. More efficient use of non-salient water, nitrogen, phosphorus, potassium, and other micro and macronutrients critical for plant growth could improve food supply while reducing resources, run-off rates, and energy.

What is needed are processes, systems and hardware for continuous, precise, in situ detection of available nutrient levels in the substrates where crops are grown. Precise real-time sensing to detect dynamic such nutrient levels with temporal and spatial resolution has the potential to significantly improve agricultural practices and reduce waste. The systems, devices, sensors and output described herein address this agricultural need by real-time detecting concentrations of such nutrients in situ at particular and traceable locations.

### BRIEF SUMMARY

In certain embodiments the subject matter described herein relates to a detection device for detecting at least one nutrient concentration in a crop cultivation substrate, comprising a test thermal sensor configured to generate one or more thermal signals, wherein the test thermal sensor comprises an enzyme, wherein the one or more thermal signals is indicative of the enthalpy of a reaction between the enzyme and a nutrient if present, wherein the nutrient is a substrate of the enzyme, and the enthalpy is indicative of the concentration of the nutrient.

In certain embodiments the subject matter described herein relates to a method of measuring a nutrient in a crop cultivation substrate, the method comprising:
receiving a thermal signal from a test thermal sensor comprising an enzyme, wherein the nutrient is a substrate of the enzyme, and receiving a thermal signal from a reference thermal sensor that does not comprise the enzyme, wherein the test thermal sensor and the reference thermal sensor are in physical communication with the crop cultivation substrate;
comparing the test thermal signal to the reference thermal signal to determine the change in enthalpy per unit of the enzyme; and,
calculating a concentration of the nutrient in the crop cultivation substrate from the change in enthalpy per unit of the enzyme.

In certain embodiments the subject matter described herein relates to a method of measuring a concentration of a nutrient, the method comprising:
detecting at least one thermal signal from an enzyme reaction with the nutrient;
determining the change in enthalpy from the at least one thermal signal;
correlating the thermal signal to a change in enthalpy per unit of the enzyme;
calculating the concentration of the nutrient from the change in enthalpy per unit of the enzyme.

In certain embodiments the subject matter described herein relates to a method of measuring a concentration of a nutrient in a crop cultivation substrate, the method comprising:

calculating the concentration by determining a change of enthalpy per unit of the enzyme from at least two thermal signals generated by a thermal sensor comprising an enzyme, wherein the thermal sensor is in physical communication with the crop cultivation substrate, wherein the nutrient is a substrate of the enzyme, and correlating the change of enthalpy per unit of the enzyme to the concentration of the nutrient, wherein the calculating and correlating are performed by a processor. These and other embodiments are described herein.

### BRIEF DESCRIPTION OF THE DRAWINGS

FIG. 1A shows a schematic of an enzyme biosensor for the detection of soil nutrients.

FIG. 1B shows the enzyme (circle) immobilized on a fiber optic cable for use in a fertilizer system as shown in FIG. 1C.

FIG. 2A shows a schematic of a fiber optics distributed temperate sensing system (FO-DTS). FIG. 2B shows a FO-DTS system for measuring nitrogen content in soil.

FIG. 3 shows the theoretical temperature changes of urease (black line) and nitrate reductase (grey line) based on substrate concentration.

FIG. 4 shows isothermal titration calorimetry (ITC) data collected after the injection of urea into a 0.028 mM solution of urease.

FIG. 5 shows a graph of temperature vs urea concentration observed from ITC testing of urease.

FIG. 6 shows ITC data collected after the injection of nitrate into a 0.4 unit/mL solution of nitrate reductase.

FIG. 7 shows theoretic and experimental temperature changes observed for the reaction of urease based on urea concentration.

FIG. 8 shows theoretic and experimental temperature changes observed for the reaction of nitrate reductase based on nitrate concentration.

FIG. 9 shows the chemical reaction of HEPES with sulfuric acid used to calibrate the lab-scale prototype following standard procedures.

FIG. 10 shows the temperature changes observed during testing of urease with the lab scale prototype.

FIG. 11 shows a schematic of a nutrient detection system comprising a fiber optical cable with an immobilized enzyme buried in a crop field and a processor for calculating nutrient concentration from thermal data.

### DETAILED DESCRIPTION

Described herein are biosensors for the in situ detection of nutrients in the crop cultivation substrates (e.g., soil or water) where crops are produced using enzymes that produce thermal signals that can be transmitted and correlated to precisely determine nutrient levels present in the crop cultivation substrate. A measurable correlation between nutrient concentration and temperature fluctuations has been observed. Biosensors, also referred to herein as detection devices, based on these parameters can provide real-time measurements of soil nutrients and provide valuable information to improve agricultural practices with more efficient use of resources while improving crop production.

In some embodiments, crop cultivation substrate measurement systems, devices, sensors and output as described herein enable growers to precisely and intensely analyze soil nutrient variations under numerous field conditions. Specifically, the systems, devices, sensors and output can assist a grower in real-time management of fertilizing decisions to achieve expected or increased crop yield response. For example, the systems, devices, sensors and output can help a grower determine an appropriate time, amount, or place (i.e., field zone area) for applying or not applying fertilizers or for selecting a specific crop seed hybrid/variety that may produce an optimal yield under the farmer's field conditions and nutrient measurements. By providing real-time measurements of level(s) of nutrient(s) present in a crop cultivation substrate, the systems, devices, sensors and output can also facilitate research development of new crop varieties with enhanced fertilizer use properties. In addition, the system can assist a user in real-time monitoring of fields with a high vulnerability for chemical pollution to handle soil nutrient loss, thus contributing to current sustainability environmental practices.

Embodiments provide the benefits of helping farmers sustainably increase productivity by applying fertilizers at the right place with right amounts. Embodiments may provide the benefit of permitting the verifying that the current fertilization program will supply adequate fertility to the current year's crop and to determine how much supplemental fertilizer is needed. Embodiments may allow measuring fields after wet seasons and/or to determine nutrient, such as nitrate, carryover after a drought. Checking fields that have different crop rotations and history of manure applications will be possible with embodiments as well. The systems, devices, sensors and output described herein address an agricultural need by real-time detecting concentrations of such nutrients in situ at particular and traceable locations, e.g., in a crop field.

The presently disclosed subject matter will now be described more fully hereinafter.

However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents and other references mentioned herein are incorporated by reference into their entirety.

### I. Overview

The development of precision agriculture technologies, which optimize systems of crop and livestock production in areas prone to uncertainty and variability will allow for the production of more food from fewer resources. The optimization of crop production with high-resolution soil monitoring maintains environmental quality and increases sustainability all the while producing the same results. Also described herein is tailoring of fertilizer application by traditional or automated methods based on the detailed information provided by nutrient sensing methods and systems. A novel “fertigation-on-demand” system to allow for nutrient application only when and where necessary will decrease the use of resources, reduce the crop production costs, and minimize environmental impacts. The biosensors disclosed herein employ an enzyme which reacts with a soil nutrient, resulting in measurable heat outputs, which can be correlated to nutrient concentration to indicate which areas of the field require more nutrients and allow for “fertigation-on-demand.” The methods disclosed herein provide the first demonstration of fiber optics distributed temperate sensing system (FO-DTS) for nutrient management, indicating it is possible to use enzyme kinetics and fiber optic thermal technology as the basis of nutrient sensing in crop fields.

Significantly improved agricultural practices can result from real-time detection and reporting of crop cultivation substrate nutrient levels with the biosensors and systems described herein. For example, leaching and volatilization cause nitrogen losses from soil to the atmosphere. Nitrate is the soil nutrient most used in plant fertilizer and is responsible for high yields of plant growth due to its role in cell formation, chlorophyll production, and amino acid/protein regulation. Most forms of nitrogen are unusable by plants, but certain microbial transformations make them available. Organic nitrogen becomes soil residue which decompose and mineralize into ammonia (NH₃). Nitrification is the formation of nitrate (NO₃⁻) from nitrite (NO₂⁻) by nitrifying bacteria. Denitrification is a biological process in which nitrate is reduced to various nitrogen species through a two-step process called dissimilation. The result of this reduction is mainly gaseous N₂, but nitr-ous/ic oxides (NₓO) are also possible. Denitrifying and nitrifying bacteria rely on soil enzymes for organic matter cycling. Many genera of bacteria contain denitrifying bacteria, such as Pseudomonas, Micrococcus, Archromobacter, Thiobacillus, Bacillus, and Aspergillus. While procedures for soil nitrate analysis in liquid media are well-established, current in situ measurements are often imprecise, noncontinuous, and/or labor intensive. The biosensors and systems described herein address these shortcomings.

Biosensors employ the intimate contact between a biorecognition element that interacts with an analyte of interest and converts the biorecognition event into a measurable signal, such as a change in temperature. Enzymes are commonly used as biorecognition elements for biomedical sensing applications and enzymatic biosensors traditionally transmit electrochemical signals for the detection and quantification of an analyte. Biosensors have great promise for real-time soil sensing to improve sustainable crop production but have not yet been implemented into agricultural practices due to a variety of limitations.

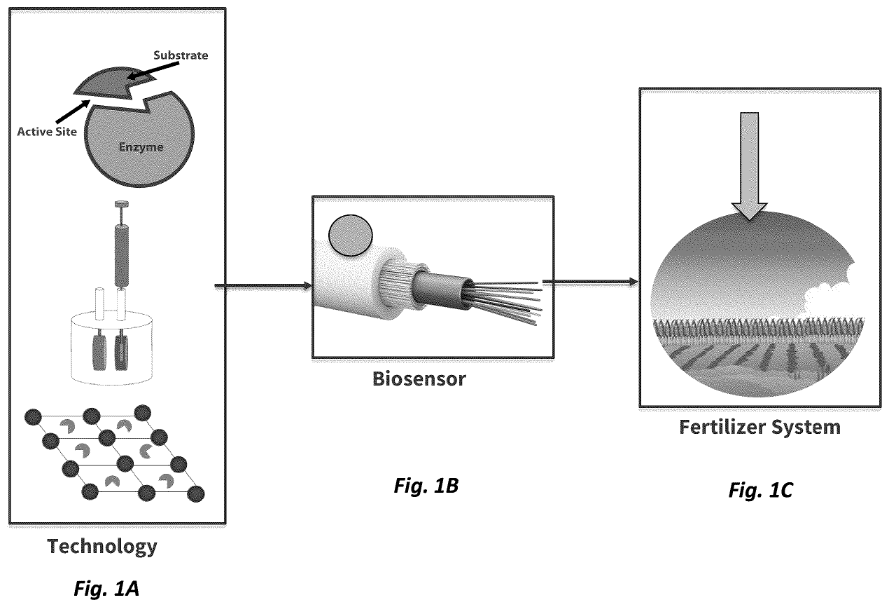

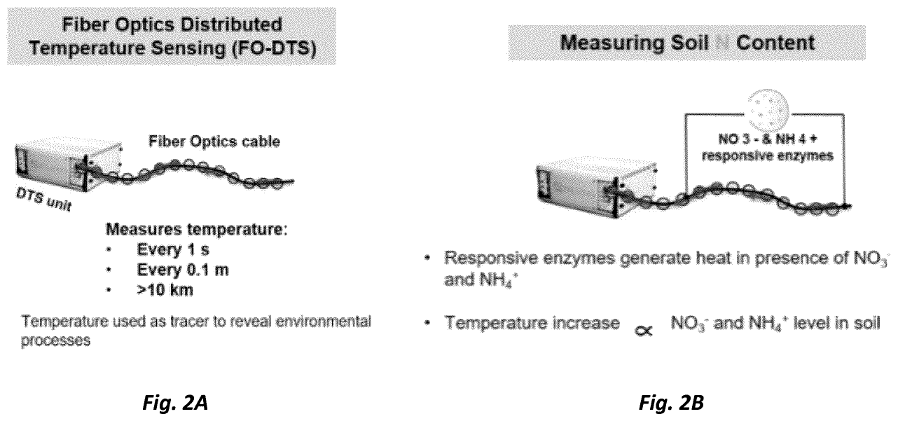

The biosensors disclosed herein utilize various enzymes that are selective for soil nutrients of interest with signal detection involving the transmission of thermal data. In some embodiments, the thermal data is transmitted by a fiber optic cable or other transmission means, such as electrical cables. The thermal signal can be measured by a thermometer, a thermocouple, and similar devices and sources. The temperature sensing apparatus with immobilized enzyme are buried in the soil near the root zone and track the thermal response of the soil. In some embodiments, the temperature readings are transmitted through the fiber optic cable and compared to a reference fiber optic cable without the immobilized enzyme and the thermal data is used to determine the concentration of the soil nutrient, as shown in FIGS. 1A-1C. In some embodiments, distributed temperature sensing (DTS) is used to measure temperature throughout the length of the fiber optic cable with high temporal and spatial frequency, as shown in FIG. 2A. Specifically, a heat transfer model and analysis system are used to estimate soil nutrient content (e.g., nitrate and/or ammonia) based on enzyme specific enthalpy data from the reaction of the particular enzyme with its specific substrate, which is the nutrient being measured (FIG. 2B).

In other embodiments, the biosensor is a probe comprising the immobilized enzyme which measures thermal data at a specific location. In some embodiments, the biosensor probe is removable. In some embodiments, thermal data is transmitted by wireless communication. In some embodiments, a plurality of biosensors are used throughout an area in active use for crop production to detect nutrient levels at different locations.

All chemical reactions either emit or absorb heat, and in theory, the thermal signal exerted by an enzyme reacting with its substrate is a function of the concentration of the substrate (e.g. soil nutrient). Enthalpy is a non-mass dependent thermodynamic quantity; thus, enthalpy is not affected by volume, concentration, or heat. As shown in Equation 1, total heat (Q) emitted is equal to the mass of the system (m) multiplied by the heat capacity (c) of the material and the change in temperature (ΔT). Equation 2 shows that enthalpy change of the reaction, accounting for number of moles of substrate (e.g., soil nutrient) is equal to the heat emitted. Equation 3 shows the theoretical calculation for reaction enthalpy, with the change in enthalpy of products and reactants as parameters.

$$Q = mc Δ T \qquad ( 1 )$$

$$Δ {H}_{rxn} * mol = Q = mc Δ T \qquad ( 2 )$$

$$Δ {H}_{rxn} = \sum m Δ {H}_{products} - \sum n Δ {H}_{reactants} \qquad ( 3 )$$

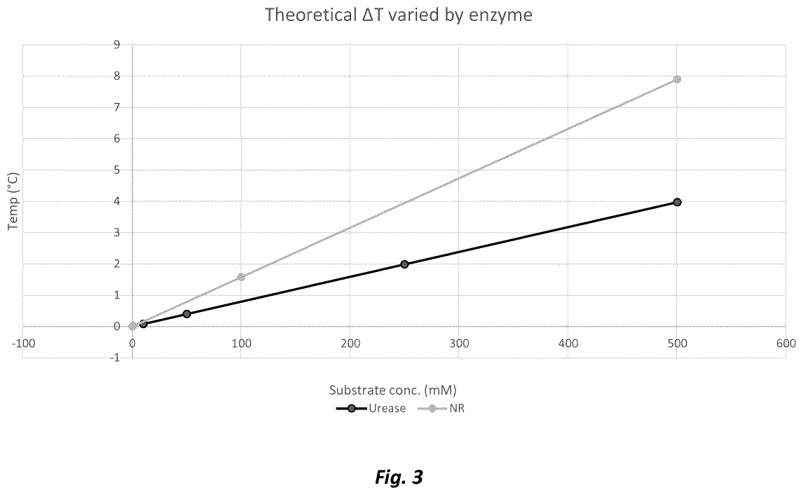

All above equations were combined to generate theoretical enthalpy of reaction for both enzymes to then compare to experimental enthalpy data. FIG. 3 shows the theoretical change in temperature of reaction for both urease and nitrate reductase reactions based on substrate concentration.

Thus, a thermal sensing enzyme-based biosensor is used to determine the concentration of its substrate (e.g., a soil nutrient). The distributed temperature sensing (DTS) method can provide frequent real time thermal data points, which are correlated to soil nutrient levels throughout an extended length of fiber optic cable. Alternatively, a plurality of biosensor probes may be placed at locations throughout a crop field and/or removable biosensor probes may be moved throughout a field. Insight on soil nutrient levels throughout a crop field over time would provide valuable data to improve crop production and fertilizer use for more sustainable agricultural practices. Continuous real-time data could be used to increase crop yields throughout different areas of a field while reducing excess fertilizer use and pollution from run-off.

### II. Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular aspects only and is not intended to be limiting of the invention. In case of a conflict in terminology, the present specification is controlling.

As used herein, the term “biosensor” refers to a detection device which uses a biological molecule (e.g., enzyme) for the detection of an analyte (e.g., nutrient).

As used herein, the term “thermal sensor” refers to a device which transmits a thermal signal. In certain embodiments, a thermal sensor can be a fiber optic cable or an electrical cable, and the like. The thermal sensor can detect heat directly or indirectly by distributed temperature sensing (DTS), a resistance temperature detector (RTD), thermometer, thermocouple, infrared (IR) sensor, or semiconductor thermal sensors, and the like.

As used herein, the term “nutrient” refers to substances that are beneficial for the growth of plants. Nutrients include but are not limited to compounds containing nitrogen (e.g., ammonia or nitrate), phosphorus (e.g., phosphate), iron, sulfur (e.g., sulfate), potassium, molybdenum, calcium, magnesium, zinc, manganese, copper, and boron. The methods and systems disclosed herein detect the concentrations of nutrients present in a crop cultivation substrate, e.g., soil or water.

As used herein, the term “crop cultivation substrate” refers to the substance on or in which a crop is grown. The crop cultivation substrate may be soil or water for a hydroponics system.

As used herein, the term “low concentration” refers to a concentration of a nutrient under a value determined by those skilled in the art as necessary for optimal crop growth.

As used herein, “change in enthalpy per unit of enzyme” refers to the change in enthalpy (ΔH) of a reaction of an enzyme with its substrate (e.g., nutrient) per micromole of the enzyme under the given conditions.

As used herein, a “soil sample” means an aliquot of any useful size of the crop cultivation substrate or an extract thereof.

As used herein, “and/or” refers to and encompasses any and all possible combinations of one or more of the associated listed items, as well as the lack of combinations when interpreted in the alternative (“or”).

As used herein, the term “about,” when referring to a measurable value such as an amount of a compound or agent of the current subject matter, dose, time, temperature, and the like, is meant to encompass variations of ±20%, ±10%, ±5%, ±1%, ±0.5%, or even ±0.1% of the specified amount.

As used herein, conditional language used herein, such as, among others, “can,” “could,” “might,” “may,” “e.g.,” and the like, unless specifically stated otherwise or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The singular forms of the articles “a,” “an,” and “the” include plural references unless the context clearly dictates otherwise. For example, the term “a protein” or “at least one protein” can include a plurality of proteins, including mixtures thereof.

Compositions or methods “comprising” or “including” one or more recited elements may include other elements not specifically recited. For example, a composition that “comprises” or “includes” a protein may contain the protein alone or in combination with other ingredients.

### III. Devices and Systems

In certain embodiments, the subject matter described herein is directed to a detection device for detecting at least one nutrient concentration in a crop cultivation substrate, comprising a test thermal sensor configured to generate one or more thermal signals, wherein the test thermal sensor comprises an enzyme, wherein the one or more thermal signals is indicative of the enthalpy of a reaction between the enzyme and a nutrient if present, wherein the nutrient is a substrate of the enzyme, and the enthalpy is indicative of the concentration of the nutrient.

In certain embodiments, the enzyme is selected from the group consisting of urease, nitrate reductase, alanine dehydrogenase, polyphosphate kinase, phosphatase, adenylyl-sulfate reductase, sulfite reductase, ferric reductase, and molybdate-binding protein ModA. In certain embodiments, the enzyme is selected from the group consisting of urease, nitrate reductase, polyphosphate kinase, adenylyl sulfate reductase, and ferric reductase. In some embodiments, the enzyme is urease or nitrate reductase.

In embodiments, the test thermal sensor is suitable for contacting a crop cultivation substrate. In certain embodiments, the crop cultivation substrate is soil or a hydroponic system. In certain embodiments, the crop cultivation substrate is soil. In certain embodiments, the test thermal sensor is a fiber optic cable.

In other embodiments, the test thermal sensor is a thermocouple, a resistance temperature detector (RTD), or a semiconductor thermal detector (i.e., thermistor). In certain embodiments, the detection device further comprises a reference thermal sensor that does not comprise an enzyme.

In certain embodiments, the detection device further comprises a second thermal sensor. In certain embodiments, the second thermal sensor is a reference thermal sensor that does not comprise an enzyme that reacts with a nutrient. In certain embodiments, the reference thermal sensor is a fiber optic cable. Fiber optic cables provide high sensitivity, rapid response times, and immunity from electrical interference, which are ideal properties for a thermal biosensor. The fiber optical cable may be of any material or length known in the art. In certain embodiments the sheath or outer jacket of the fiber optic cable is made of medium- and/or high-density polyethylene.

In embodiments, the nutrient comprises nitrogen, phosphorus, sulfur, potassium, iron, or molybdenum. In certain embodiments, the nutrient is selected from the group consisting of urea, nitrate, ammonia, phosphate, and sulfate. In certain embodiments, the nutrient is urea or nitrate. Soil nutrients and corresponding enzymes for their detection are provided in Table 1.

TABLE 1
Soil nutrients and enzymes for their detection.

| Soil Nutrient | Enzyme |
|---|---|
| urea | urease |
| nitrate | nitrate reductase |
| ammonia | alanine dehydrogenase |
| phosphate | polyphosphate kinase |
| iron | ferric reductase |
| sulfur | APS reductase or sulfite reductase |
| molybdenum | molybdate-binding protein ModA |

Ureases (EC 3.5.1.5) are nickel-containing metalloenzymes which catalyze the hydrolysis of urea to ammonia shown in Equation 4. Ureases use sodium phosphate and are found in bacteria, fungi, algae, plants and some invertebrates. In one embodiment, urease is derived from Canavalia ensiformis, a legume.

Urea+H₂O→CO₂+2NH₃ (4)

Nitrate reductases (EC 1.7.99.4) are molybdoenzymes which catalyze the reduction of nitrate (NO₃⁻) to nitrate (NO₂⁻) as shown in Equation 5 by a two-electron transfer. Nitrate reductase is expressed in bacteria, fungi, algae and some higher plants using potassium phosphate, flavin adenine dinucleotide (FAD), and nicotinamide adenine dinucleotide phosphate (NADPH). In one embodiment, nitrate reductase is derived from Aspergillus niger, a fungal mold.

NADPH+NO₃⁻→NADP+NO₂⁻+H₂O (5)

Alanine dehydrogenases (EC 1.4.1.1) catalyze a reversible deamination of L-alanine, forming pyruvate and ammonia, as shown in Equation 6.

L-alanine+H₂O+NAD⁺ [symbol] pyruvate+NH₃+NADH+H⁺ (6)

Polyphosphate kinases (EC 2.7.4.1) catalyze the formation of polyphosphate from ATP, as shown in Equation 7.

ATP+(phosphate)ₙ [symbol] ADP+(phosphate)ₙ₊₁ (7)

Ferric reductases (EC 1.16.1.7) catalyze the reduction of ferric iron (Fe³⁺) to ferrous iron (Fe²⁺) using NADH.

Adenylyl-sulfate reductases (EC 1.8.99.2) catalyze the reduction of adenylyl-sulfate/adenosine-5′-phosphosulfate (APS) to sulfite with an electron donor cofactor.

Sulfite reductases (EC 1.8.99.1) catalyze the reduction of sulfite to hydrogen sulfide with an electron donor, such as NADPH.

Molybdate-binding protein ModA is a molybdenum-specific periplasmic binding protein that binds molybdate at high affinity.

In certain embodiments, detection of the thermal signal along the one or more thermal sensors is measured by distributed temperature sensing (DTS), resistance temperature detectors (RTD), thermocouples, thermometers, infrared (IR) sensors, or semiconductor thermal sensors. In certain embodiments, the test thermal sensor comprises a fiber optic cable and the test thermal signal is measured by DTS along the test thermal sensor and the reference thermal signal is measured by DTS along the reference thermal sensor.

In certain embodiments, the thermal sensor comprises a fiber optic cable, and a DTS system (e.g., SILIXA XT®) with sampling and temporal resolutions, e.g., 0.125 meter and 1 second, which can be used to monitor the temperature over the whole length of the fiber optic cable. An internal calibration algorithm can be used to generate the temperature profiles along the fiber optic cables. An external calibration algorithm that is run on a processor that is physically connected or remotely connected to the DTS system (or cloud computing), can also be used to calibrate the thermal signal. Similarly an algorithm to convert the thermal signal into concentration of nutrient can be run internally to the DTS system, or remotely on another computing system.

In embodiments, the concentration of the nutrient is determined from a change in enthalpy per unit of the enzyme.

In embodiments, the enzyme is immobilized to the test thermal sensor by adsorption, covalent bonding, cross-linking, encapsulation, or entrapment. In embodiments, the enzyme can be immobilized on the fiber optic cable by any means known in the art. The immobilization technique is selected to protect the enzyme and provide a microenvironment compatible for the reaction. In certain embodiments the enzyme is immobilized in a gel matrix. In certain embodiments, the enzyme is immobilized by entrapment in an alginate gel or a gelatin gel. In certain embodiments, the enzyme is immobilized by cross-linking with a polymer. In certain embodiments, the enzyme is immobilized with a temperature-sensitive hydrogel, a polyaniline-nickel oxide (PANI-NiO) nanocomposite, or holey optical fibers. In certain embodiments, the enzyme is encapsulated within an electrospun material. In certain embodiments, the electrospun material is a sheath (e.g., outer jacket) on the test thermal sensor.

In certain embodiments, the enzyme is immobilized with a cofactor and/or buffer. In certain embodiments the cofactor and/or buffer for the enzymatic reaction is NADH, NADPH, FAD, NAD, Fd, Tris-HCl buffer, potassium phosphate, sodium phosphate, sodium carbonate, sodium bicarbonate, disulfonic acid, oxalyl chloride, dimethylformamide, dichloromethane, diisopropylethalamine, ethyl acetate, dithionite, methyl viologen. In certain embodiments, the enzyme is immobilized with a buffer. In certain embodiments, immobilization of the cofactor is achieved by retention via electrostatic repulsion from charged membranes, with microcapsules to capture small pored cofactors, and/or by chemically modifying cofactors to increase their size. In certain embodiments, immobilization supports comprise natural polymers, synthetic polymers, and/or inorganic materials such as alginate, cellulose, starch, ceramics, glass, and charcoal. In certain embodiments, enzyme immobilization is confirmed using UV spectrophotometry.

In certain embodiments the enzyme is immobilized throughout the entire length of the thermal sensor, e.g., a fiber optic cable. In other embodiments, the enzyme is immobilized on selected portions of the thermal sensor, e.g., a fiber optic cable.

In certain embodiments, the enzyme is immobilized within one or more cartridges in physical communication with the test thermal sensor. In certain embodiments, a cofactor and/or buffer is immobilized with the cartridge. In certain embodiments, the detection device comprises at least two cartridges, wherein each of the cartridges is separated from another cartridge at a distance from about 0 cm to about 10,000 m along the test thermal sensor. In certain embodiments, the detection device comprises at least two cartridges, wherein each of the cartridges is separated from another cartridge at a distance from about 5 cm to about 100 cm. In some embodiments, spacing between cartridges is uniform. In some embodiments, spacing between cartridges is not uniform. In some embodiments, the cartridges are replaceable and may be exchanged with new cartridges.

In some embodiments, the detection device is a probe comprising the enzyme immobilized on a thermal sensor. A plurality of probes may be used to obtain a plurality of thermal signals to determine nutrient concentrations for a plurality of locations. In certain embodiments, the enzyme is immobilized on at least a portion of a thermal sensor, such as a thermocouple, a resistance temperature detectors (RTD), or semiconductor thermal sensor (i.e., thermistor). In a non-limiting example, the detection device may comprise the enzyme that reacts with a nutrient immobilized on a region at the end of an RTD temperature sensor, such as a RTD1000 ohm sensor wire from Process Technology.

Provided herein are detection devices for detecting urea or nitrate concentration in soil, comprising a test thermal sensor configured to generate one or more thermal signals, wherein the test thermal sensor comprises urease or nitrate reductase, wherein the one or more thermal signals is indicative of the enthalpy of a reaction between urease and urea or nitrate reductase and nitrate if present, and the enthalpy is indicative of the concentration of the nutrient. The detection device may comprise any of the test thermal sensors and reference thermal sensors described herein. The urease or nitrate reductase may be immobilized on the test thermal sensor using any of the methods described herein. The detection device may process and/or transmit the thermal signals using any of the methods disclosed herein.

In some embodiments, the thermal signals are transmitted by wireless communication. Any technology known in the art may be used for the transmission and processing of the thermal signals.

In embodiments, the thermal signal is collected and analyzed by a processor by any means known in the art. In certain embodiments a Python program is used to retreat and process DTS data from fiber optic detection device.

In certain embodiments, the enthalpy change per unit of enzyme is characterized for a nutrient and its enzyme by calorimetry, such as isothermal titration calorimetry.

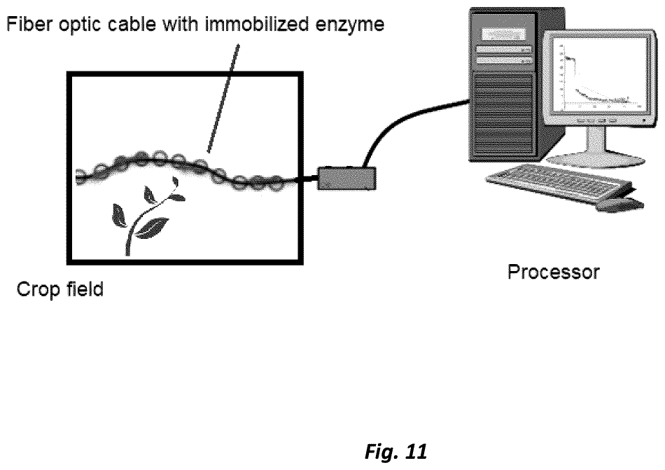

A schematic of a nutrient detection system comprising a fiber optical cable with an immobilized enzyme buried in a crop field and a processor for calculating nutrient concentration from thermal data, which includes temperature readouts and enthalpy calculations, is shown in FIG. 11.

In embodiments the detection device further comprises a processor and system memory, wherein the processor and system memory are configured to implement a method of identifying a change in enthalpy per unit of enzyme from the one or more thermal signals, and correlating the change in enthalpy per unit of enzyme to generate an output, where the output is an indication of the concentration of the nutrient that can be present in the crop cultivation substrate.

In certain embodiments, the techniques described herein are implemented by one or more computing devices. The computing devices may be hard-wired to perform the techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such computing devices may also combine custom hard-wired logic, ASICs, or FPGAs with custom programming to accomplish the techniques. The computing devices may be desktop computer systems, portable computer systems, handheld devices, networking devices or any other device that incorporates hard-wired and/or program logic to implement the techniques.

In certain embodiments, the computer system can include a bus or other communication mechanism for communicating information, and a hardware processor coupled with bus for processing information. Hardware processor may be, for example, a general purpose microprocessor. The computer system can also include a main memory, such as a random access memory (RAM) or other dynamic storage device, coupled to bus for storing information and instructions to be executed by the processor. The main memory also may be used for storing temporary variables or other intermediate information during execution of instructions to be executed by the processor. Such instructions, when stored in non-transitory storage media accessible to the processor, render the computer system into a special-purpose machine that is customized to perform the operations specified in the instructions.

The computer system can further include a read only memory (ROM) or other static storage device coupled to bus for storing static information and instructions for the processor. A storage device, such as a magnetic disk, optical disk, or solid-state drive can be provided and coupled to bus for storing information and instructions. The computer system may be coupled via bus to a display for displaying information to a computer user. An input device, including alphanumeric and other keys, is coupled to bus for communicating information and command selections to the processor. Another type of user input device is a cursor control, such as a mouse, a trackball, or cursor direction keys for communicating direction information and command selections to the processor and for controlling cursor movement on the display.

The computer system can also include a communication interface coupled to bus. The communication interface provides a two-way data communication coupling to a network link that is connected to a local network. For example, the communication interface may be an integrated services digital network (ISDN) card, cable modem, satellite modem, or a modem to provide a data communication connection to a corresponding type of telephone line. As another example, the communication interface may be a local area network (LAN) card to provide a data communication connection to a compatible LAN. Wireless links may also be implemented. In any such implementation, the communication interface sends and receives electrical, electromagnetic or optical signals that carry digital data streams representing various types of information. The network link typically provides data communication through one or more networks to other data devices. For example, the network link may provide a connection through local network to a host computer or to data equipment operated by an Internet Service Provider (ISP). The ISP in turn provides data communication services through the Internet. The computer system can send messages aid receive data, including program code, through the network(s), network link and communication interface. In the Internet example, a server might transmit a requested code for an application program through Internet, ISP, local network and communication interface.

In certain embodiments, the subject matter described herein is directed to a machine-readable storage medium storing instructions that when executed by a processor, cause the processor to execute a method of measuring the concentration of a nutrient present in a crop cultivation substrate, the method comprising: communicating with a detecting device that is communicatively coupled either directly or indirectly to the processor, the detecting device comprising a test thermal sensor configured to generate one or more thermal signals, wherein the test thermal sensor comprises an enzyme, wherein the one or more thermal signals is indicative of the enthalpy of a reaction between the enzyme and a nutrient if present, wherein the nutrient is a substrate of the enzyme, and the enthalpy is indicative of the concentration of the nutrient; and, receiving a signal from the detection device; and, calculating a concentration level of the nutrient in the crop cultivation substrate from the change in enthalpy per unit of the enzyme. The signal received from the detection device can include a test thermal signal and a reference thermal signal.

In certain embodiments, a crop cultivation substrate measurement system comprises a detecting device as described herein, and a processor, such as a computer with a custom-made software for controlling the entire process.

### IV. Methods

The methods described herein can be utilized with any of the described systems and devices.

In certain embodiments, the methods described herein are directed to receiving a thermal signal from a test thermal sensor comprising an enzyme, wherein the nutrient is a substrate of the enzyme, and receiving a thermal signal from a reference thermal sensor that does not comprise the enzyme, wherein the test thermal sensor and the reference thermal sensor are in physical communication with the crop cultivation substrate;
comparing the test thermal signal to the reference thermal signal to determining the change in enthalpy per unit of the enzyme; and,
calculating a concentration of the nutrient in the crop cultivation substrate from the change in enthalpy per unit of the enzyme.

In certain embodiments, the thermal signals are measured by DTS, and the thermal sensors comprise a fiber optic cable. The thermal sensors can further comprise a transducer.

In certain embodiments, a plurality of probes comprising the enzyme immobilized on a thermal sensor are disposed at a plurality of locations throughout the crop cultivation substrate.

In some embodiments, the detection device is placed in contact with an area in active use for crop production.

In certain embodiments, the test thermal sensor and the reference thermal sensor are disposed on an area of from 1 square centimeter to 1000 hectares.

In certain embodiments, the test thermal sensor and the reference thermal sensor are disposed in a pattern along the area. In certain embodiments, the pattern is one or more rows, a grid, a checkered or circular pattern.

In certain embodiments, the enzyme is selected from the group consisting of urease, nitrate reductase, alanine dehydrogenase, polyphosphate kinase, phosphatase, adenylyl-sulfate reductase, sulfite reductase, ferric reductase, and molybdate-binding protein ModA.

In certain embodiments, a method of measuring a concentration of a nutrient comprises detecting at least one thermal signal from an enzyme reaction with the nutrient; determining the change in enthalpy from the at least one thermal signal; correlating the thermal signal to a change in enthalpy per unit of the enzyme; and calculating the concentration of the nutrient from the change in enthalpy per unit of the enzyme. In certain embodiments, the thermal signal is measured by isothermal titration calorimetry. In certain embodiments, the thermal signal is measured with a thermocouple.

In certain embodiments, a method of measuring a concentration of a nutrient in a crop cultivation substrate comprises calculating a change of enthalpy per unit of the enzyme from at least two thermal signals generated by a test thermal sensor comprising an enzyme, wherein the thermal sensor is in physical communication with the crop cultivation substrate, wherein the nutrient is a substrate of the enzyme, and correlating the change of enthalpy per unit of the enzyme to the concentration of the nutrient, wherein the calculating and correlating are performed by a processor. In certain embodiments, the enzyme is immobilized on the test thermal sensor at a known concentration. In certain embodiments, the measuring is real-time and/or continuous.

In certain embodiments, a method of increasing the efficiency of crop production in a field, comprises receiving a measurement of one or more nutrient concentrations in the field with said detection device, identifying at least one low concentration of the one or more nutrient concentrations in the field; and, fertilizing one or more areas of the field to increase the concentration of the nutrient. In certain embodiments, the method is repeated at a frequency for a period of time.

In certain embodiments, a method of increasing the efficiency of fertilizer use in a field comprises measuring one or more nutrient concentrations in the field with said detection device, identifying at least one low concentration of the one or more nutrient concentrations in one or more areas of the field; and fertilizing with the one or more nutrients of low concentration; and/or fertilizing one or more areas of the field with the low concentration of the one or more nutrients; and/or applying a minimum quantity of fertilizer to increase the low concentration of the one or more nutrients; and/or repeating an application of fertilizer at a frequency to increase the low concentration of the one or more nutrients.

In certain embodiments, a method of reducing fertilizer run-off in a field comprises measuring one or more nutrient concentrations in the field with said detection device, identifying at least one low concentration of the one or more nutrient concentrations in one or more areas of the field; and limiting fertilizer application to the one or more nutrients of low concentration; and/or limiting fertilizer application to the one or more areas of the field with the low concentration of the one or more nutrients; and/or minimizing a quantity of fertilizer applied to increase the low concentration of one or more nutrients; and/or minimizing a frequency of fertilizer application to increase the low concentration of one or more nutrients.

In certain embodiments, the thermal sensor is buried in soil of a crop field. In some embodiments the thermal sensor is buried near the root zones. In some embodiments, the thermal sensor is buried at a depth of about 5 cm to a depth of about 5 m. In some embodiments, transducer elements are installed at several different depths. In one embodiment, thermal sensors are installed at depths of 5 cm, 10 cm, and 15 cm. In one embodiment, thermal sensors are installed at depths of 30 cm, 60 cm, and 90 cm. In some embodiments, the thermal sensor is oriented back and forth in rows, in a grid, in a circular pattern, or in a checkered pattern.

In one embodiment, a center-pivot system is used to apply fertilizer on an as-needed basis based on calculated soil nutrient levels. The near-real-time readouts from the biosensors disclosed herein allow for a continuous system using a dynamic feedback loop to maintain soil nutrient levels by controlling fertilizer application in crop fields. In certain embodiments, an integrated feedback loop for soil fertilization uses a proportional-integral-derivative (PID) control. A feedback dashboard could include baseline soil nutrient data based on cover crops, soil type, weather, and/or historical data.

### EXAMPLES

### Example 1: Quantifying Enthalpy of Urease and Nitrate Reductase

Testing of the enzymatic reactions of urease and nitrate reductase was performed to measure thermal output from the reactions to establish and confirm a correlation between substrate concentration and enthalpy of reaction. The reaction enthalpy change (ΔHᵣₓₙ) per unit of enzyme is first characterized and the sensitivity of the biosensor was assessed by analyzing enthalpy data to draw a conclusive correlation between nutrient concentration and temperature of reaction. The average temperature response per unit of enzyme is quantified given the change in temperature (ΔT) recorded by the thermal biosensor and is directly proportional to the enthalpy change.

First, nano-isothermal titration calorimetry (ITC) was utilized to measure the heat expelled or absorbed upon titration of a set injection volume of solution of the substrate (e.g., urea or nitrate) into a sample cell containing an enzyme solution and buffers. The enthalpy generated from this reaction is then compared to the enthalpy of a controlled reference cell, where generated heat is proportional to amount of formed complex. Data fitting data provides results on stoichiometry (v), association constant (Kₐ), and enthalpy (H).

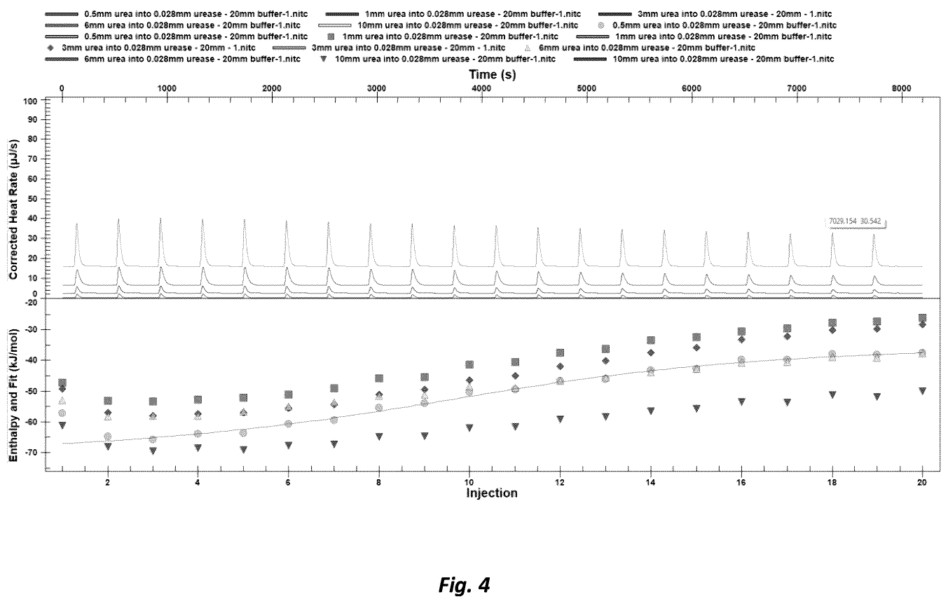

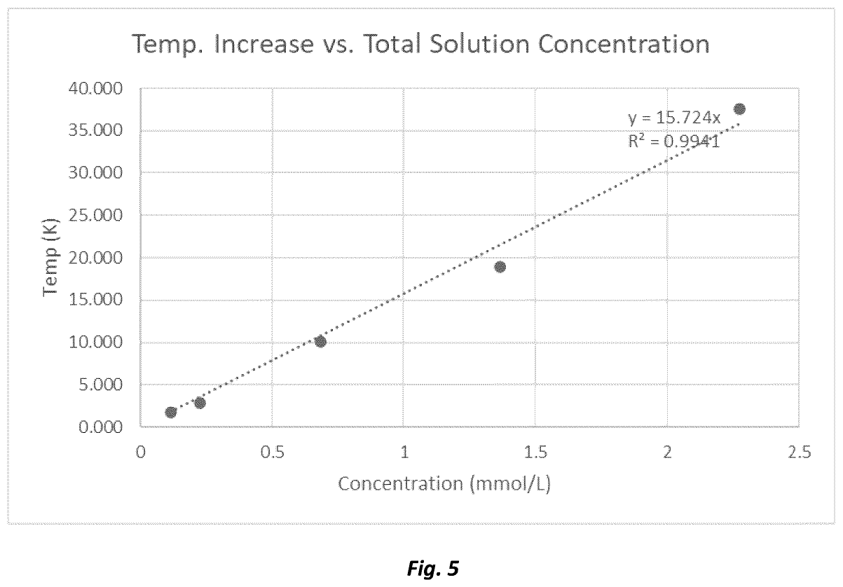

Following standard ITC testing procedures, testing was performed on a TA Instruments nano-isothermal titration calorimeter. Four rounds of testing was performed with a constant 0.028 mM concentration of urease with urea buffer concentrations of 0.5, 1, 3, 6, and 10 mM, as shown in FIG. 4. Post-testing data analysis was conducted via NanoAnalyze to determine enthalpy and temperature change per reaction. The linear response observed in the temperature change of the urease reaction as the total solution concentration increases as expected (FIG. 5).

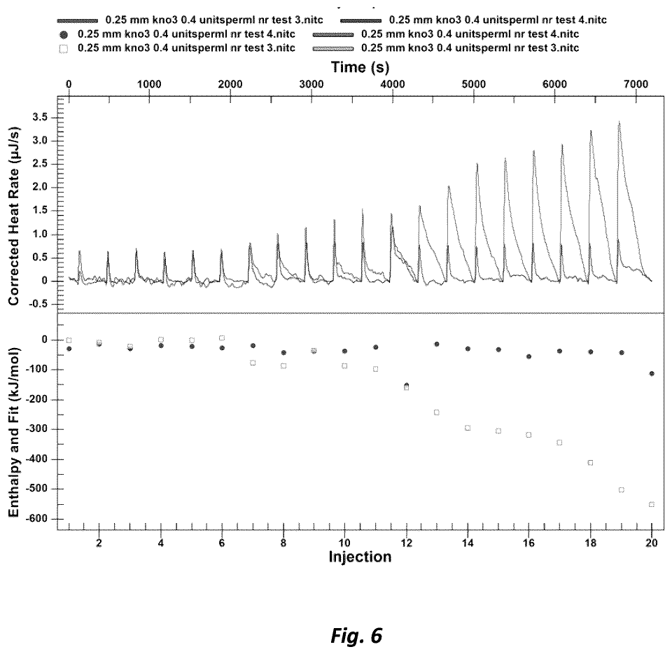

Additionally, nitrate reductase underwent three rounds of ITC testing with a constant concentration of 0.4 units/mL of enzyme with 0.05, 0.25, and 0.5 mM concentrations of nitration buffer, as shown in FIG. 6.

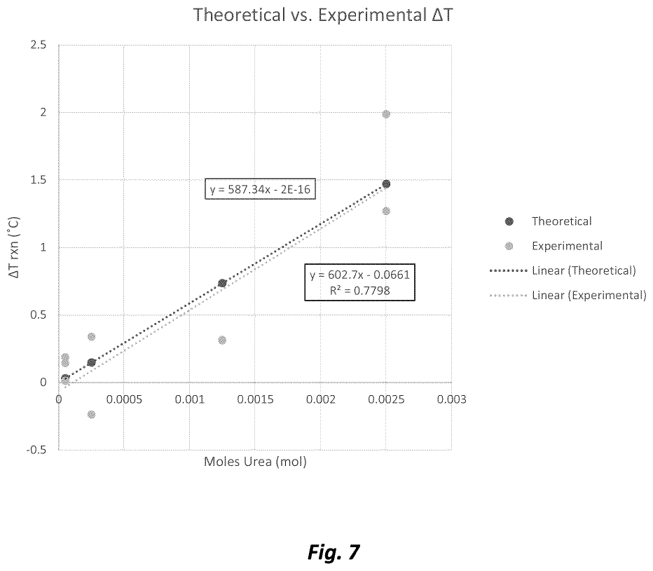

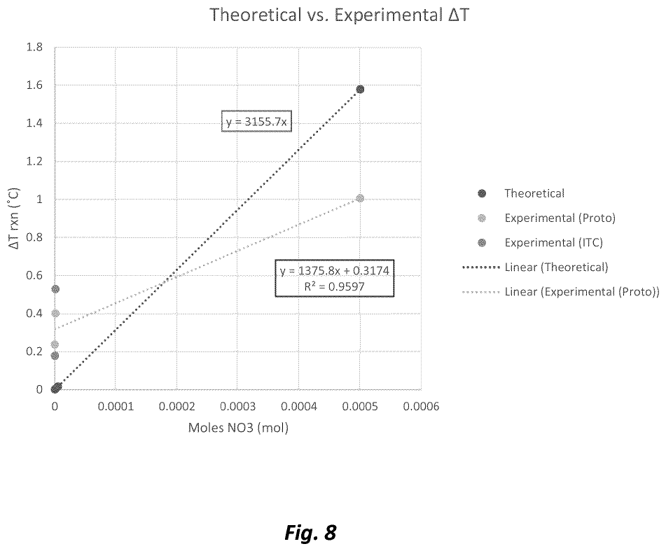

The experimental ΔT values for both urease (FIG. 7) and nitrate reductase (FIG. 8) showed a strong correlation between nutrient concentration and temperature of reaction, as expected with theoretical ΔT values.

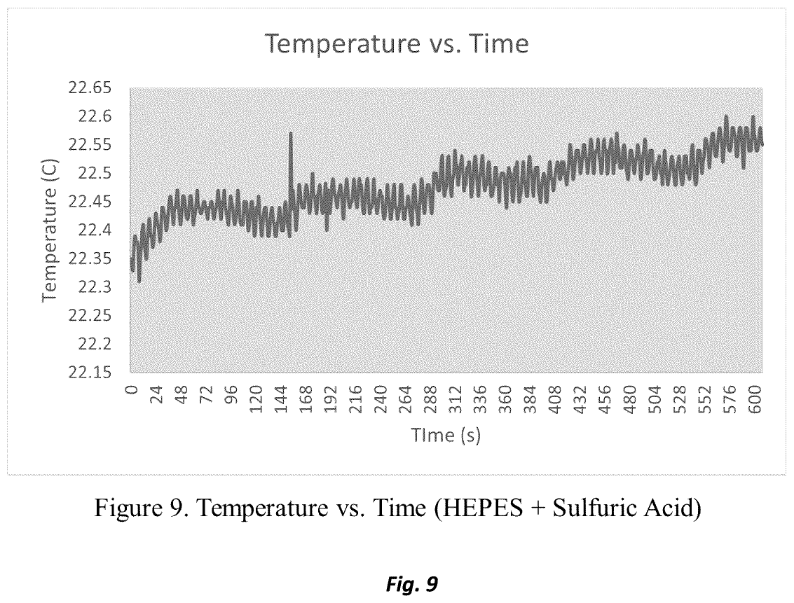

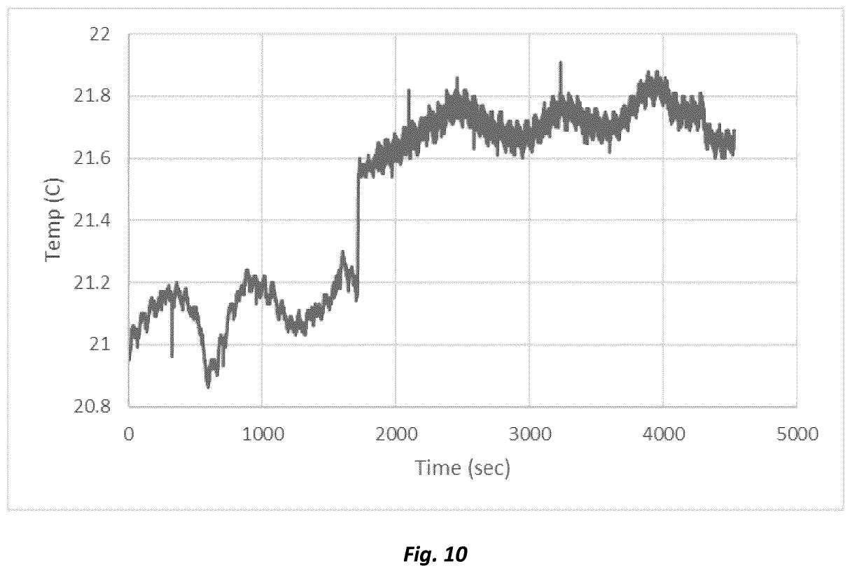

The urease reaction was further tested with a lab-scale prototype to confirm the temperature-concentration correlations outside of the nanocalorimeter. The prototype included a foam cooler, thermocouple wire, a stir bar mechanism, and a data logger. Multiple thermocouple types were compared and a Type T thermocouple with a range of −200-200° C., precision of =/−0.5° C., and a response time of 0.002 seconds was chosen for use. A simple chemical test with sulfuric acid and HEPES was performed following standard procedures to calibrate the prototype using an ice bath to compare heat per injection (J), as shown in FIG. 9. The protype was then used to detect temperature changes during the reaction of urea with urease over various concentrations of both. Post-testing analysis in Excel showed the temperature increased upon the addition of urea into the solution with urease and results show linearity between change in temperature of reaction and concentration of substrate. (FIG. 10).

Efforts have been made to ensure accuracy with respect to numbers used (e.g., amounts, temperature, etc.) but some experimental errors and deviations should be accounted for.

One skilled in the art will recognize many methods and materials similar or equivalent to those described herein, which could be used in the practicing the subject matter described herein. The present disclosure is in no way limited to just the methods and materials described.

Unless defined otherwise, technical, and scientific terms used herein have the same meaning as commonly understood by one of ordinary skill in the art to which this subject matter belongs.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

1. A detection device for detecting at least one nutrient concentration in a crop cultivation substrate, comprising a test thermal sensor configured to generate one or more thermal signals, wherein the test thermal sensor comprises an enzyme, wherein the one or more thermal signals is indicative of the enthalpy of a reaction between the enzyme and a nutrient if present, wherein the nutrient is a substrate of the enzyme, and the enthalpy is indicative of the concentration of the nutrient.

2. The detection device of claim 1, wherein the enzyme is selected from the group consisting of urease, nitrate reductase, alanine dehydrogenase, polyphosphate kinase, phosphatase, adenylyl-sulfate reductase, sulfite reductase, ferric reductase, and molybdate-binding protein ModA.

3.-5. (canceled)

6. The detection device of claim 1, wherein the crop cultivation substrate is soil or a hydroponic system.

7. (canceled)

8. The detection device of claim 1, wherein the test thermal sensor comprises a transducer, and a fiber optic cable or a cable comprising an electrically conductive material.

9. The detection device of claim 1, further comprising a second thermal sensor configured to generate one or more thermal signals.

10. The detection device of claim 9, wherein the second thermal sensor is a reference thermal sensor that does not comprise an enzyme, wherein the nutrient is a substrate of the enzyme.

11. (canceled)

12. The detection device of claim 1, further comprising a processor and system memory, wherein the processor and system memory are configured to implement a method of identifying a change in enthalpy per unit of enzyme from the thermal signal of the test thermal sensor compared to the thermal signal of the reference thermal sensor, and generating an output that is indicative of the concentration of a nutrient in the crop cultivation substrate.

13. (canceled)

14. The detection device of claim 12, wherein the nutrient is selected from the group consisting of urea, nitrate, ammonia, phosphate, iron, molybdate, and sulfate.

15. The detection device of claim 1, wherein the thermal signal is measured by distributed temperature sensing (DTS), resistance temperature detectors (RTD), infrared (IR) sensors, semiconductor thermal sensors, thermocouples, or thermometers.

16. The detection device of claim 15, wherein the test thermal sensor comprises a fiber optic cable configured to measure the thermal signal by DTS, and the reference thermal sensor comprises a fiber optic cable configured to measure the thermal signal by DTS.

17. The detection device of claim 1, wherein the enzyme is immobilized on the test thermal sensor.

18. The detection device of claim 17, wherein the enzyme is immobilized to the test thermal sensor by adsorption, covalent bonding, cross-linking, encapsulation, or entrapment.

19.-24. (canceled)

25. A method of measuring a nutrient in a crop cultivation substrate, the method comprising:
receiving a thermal signal from a test thermal sensor comprising an enzyme, wherein the nutrient is a substrate of the enzyme, and receiving a thermal signal from a reference thermal sensor that does not comprise the enzyme, wherein the test thermal sensor and the reference thermal sensor are in physical communication with the crop cultivation substrate;
comparing the test thermal signal to the reference thermal signal to determine the change in enthalpy per unit of the enzyme; and,
calculating a concentration of the nutrient in the crop cultivation substrate from the change in enthalpy per unit of the enzyme.

26. The method of claim 25, wherein the thermal signals are measured by DTS, and the thermal sensors comprise a fiber optic cable.

27.-29. (canceled)

30. The method of claim 25, where the enzyme is selected from the group consisting of urease, nitrate reductase, alanine dehydrogenase, polyphosphate kinase, phosphatase, adenylyl-sulfate reductase, sulfite reductase, ferric reductase, and molybdate-binding protein ModA.

31.-38. (canceled)

39. The detection device of claim 1, wherein the test thermal sensor is a thermocouple, resistance temperature detector (RTD), or a semiconductor thermal sensor.

40. (canceled)

41. The method of claim 25, wherein the thermal signals are measure by a thermocouple, a resistance temperature detectors (RTD), or a semiconductor thermal sensors.

42. The method of claim 41, wherein the method comprises receiving a plurality of thermal signals from a plurality of test thermal sensors.

43. The method of claim 25, wherein the thermal signals are transmitted by wireless communication.

44. A detection device for detecting urea or nitrate concentration in soil, comprising a test thermal sensor configured to generate one or more thermal signals, wherein the test thermal sensor comprises urease or nitrate reductase, wherein the one or more thermal signals is indicative of the enthalpy of a reaction between urease and urea or nitrate reductase and nitrate if present, and the enthalpy is indicative of the concentration of the nutrient.
